(19) United States
(12) Patent Application Publication
(10) Pub. No.: US 2006/0068020 A1
(43) Pub. Date: Mar. 30, 2006

(54) Encapsulated pharmaceutical agents

(76) Inventors: Ian W. Cottrell, Basking Ridge, NJ (US); Albert Ahn, Short Hills, NJ (US); Richard Fisher, Brookville, OH (US)

Correspondence Address: STROOCK & STROOCK & LAVAN LLP, 180 MAIDEN LANE, NEW YORK, NY, 10038, US

(21) Appl. No.: 11/228,207

(22) Filed: Sep. 16, 2005

Related U.S. Application Data
(60) Provisional application No. 60/612,658, filed on Sep. 24, 2004.

(51) Int. Cl. A61K 31/7048; A61K 31/4965; C07D 239/42

(52) U.S. Cl. 424/490; 514/28; 514/266.22; 514/255.06; 514/249; 514/256

(57) ABSTRACT

The present invention provides stable lipid-based pharmaceutical formulations containing ivermectin, praziquantel and pyrantel pamoate, and methods for preparing the formulations. Further provided is a method of treating helminthiasis of mammals, which comprises administering a pharmaceutical composition that is highly effective against helminths, particularly tapeworm, hookworm, roundworm and heartworm of domestic animals and farm animals.

This application claims the benefit of U.S. Provisional Application No. 60/612,658, filed Sep. 24, 2004.

### BACKGROUND OF INVENTION

The invention relates generally to anthelmintic formulations which can have significant parasiticidal activity as anthelmintics in animal health and more particularly to solid anthelmintic formulations containing ivermectin.

Active ingredients of anthelmintics and their methods of formation in accordance with preferred embodiments of the invention are discussed in U.S. Pat. Nos. 3,502,661, 4,001,411 and 4,199,569, pending U.S. Ser. Nos. 10/637,807 and 10/800,407, and PCT/US04/025,005 entitled “Improved Anthelmintic Formulations, filed Aug. 3, 2004, the contents of which are incorporated herein by reference.

It is often beneficial, under certain circumstances, to include multiple drugs in the same formulation in order to target a wider variety of parasites. For example, a multidrug formulation may be useful in overcoming problems seen with single drug resistance. The inclusion of greater than one anthelmintic in the formulations discussed herein may have an increased likelihood of eliminating a particular helminth that is resistant to other included anthelmintic compounds. Accordingly, even if the helminth is resistant to one or two of the ingredients, it is likely that at least one of the other ingredients will be effective at eliminating the helminth in question.

The disease or group of diseases described generally as helminthiasis is due to infestation of an animal host with parasitic worms known as helminths. Helminthiasis is a prevalent and serious economic problem in domesticated animals such as swine, sheep, horses, cattle, goats, dogs, cats and poultry. Among the helminths, the group of worms described as nematodes causes widespread and often times serious infection in various species of animals. Still other parasites may be located in other tissues and organs of the body such as the heart and blood vessels, subcutaneous and lymphatic tissue and the like. The parasitic infections known as helminthiasis lead to anemia, malnutrition, weakness, weight loss, severe damage to the walls of the intestinal tract and other tissues and organs and, if left untreated, may result in death of the infected host.

Examples of particularly desirable parasitical agents include ivermectin and praziquantel. However, ivermectin is hygroscopic and therefore tends to be undesirably unstable. It has also been determined that ivermectin is unstable in both acidic and basic solutions and is susceptible to photodegradation and oxidative degradation. Accordingly, it is very difficult to prepare a solid composition, such as a powder, tablet or pellet, containing ivermectin without having to resort to using a large amount of filler material to make up the bulk of the solid in order to maintain the integrity of the compound. Even then, degradation problems can exist.

This problem is compounded when additional actives are intended to be included in the same formulation, as the interaction of ivermectin with other actives can result in an unstable formulation. Thus, while it is desirable to formulate a composition containing ivermectin and other parasitical agents such as praziquantel, it can be difficult to formulate a stable multidrug composition that can be stored at room temperature for reasonable amounts of time.

U.S. Pat. No. 6,340,672 discloses a liquid solvent system for multiple anthelmintics. However, it does not provide a solid formulation or address the problem of instability. U.S. Pat. No. 4,597,969 discloses mixing an active compound such as ivermectin with a second ingredient and spray granulating the resulting formulation with alginic acid. However, alginic acid, being negatively charged, can bind to compounds having a positive charge such as pyrantel, which could impact the bioavailability of the anthelmintics.

The pending U.S. Ser. No. 10/637,807 discloses a solid anthelmintic formulation that achieves stability improvements through spray granulation with a neutral carrier instead of alginic acid. While this method improves stability, spray granulation methods can be difficult to scale-up for commercial production.

Accordingly, it is desirable to provide improved formulations and improved methods of preparing stable multidrug anthelmintic formulations which can be formed into a solid or tablet of optimal size, palatable to animals and can be easily administered to the affected animal.

### SUMMARY OF THE INVENTION

Generally speaking, in accordance with the invention, a pharmaceutical formulation and method of preparation is provided for use in the treatment of helminthiasis of mammals, and particularly tapeworm, hookworm, roundworm and heartworm of domestic animals and farm animals. Accordingly, the present invention provides a method of treating helminthiasis in mammals, which method comprises administering to the mammal in need thereof, an anthelmintically effective amount of a pharmaceutical formulation of the invention. The present invention also provides a method for preparing pharmaceutical formulations containing drugs, which tend to react adversely with other desirable components, such as ivermectin, in combination with other active compositions such as hexahydropyrazinoisoquinolines and anthelmintic pyrimidines such as tetrahydropyrimidines. Examples of these include, for example, praziquantel and pyrantel pamoate, respectively. Formulations in accordance with the invention can remain stable when stored at room temperature for over one month, and typically, much longer.

One preferred method involves encapsulating at least one active ingredient, preferably ivermectin, praziquantel or both, prior to mixing the active ingredients into a lipid containing composition. The encapsulation of ivermectin, praziquantel or both ivermectin and praziquantel prevents ivermectin and praziquantel from interacting with each other. Once encapsulated, the dried ingredients including the encapsulated and non-encapsulated ingredients, can be mixed with melted lipid to form a homogeneous liquid lipid suspension. The lipid suspension containing the active ingredients is formed into the desired dosage form by molding or pouring the suspension into a suitable container or mold and cooled to a solid form.

As used herein, terms such as melted, liquid, solid and so forth are not given their strict technical meaning because many lipid containing materials do not have definite melting and freezing points. A skilled artisan will appreciate that many lipids are solid at approximately room temperature, yet are liquid or pourable at a higher temperature. In the present invention, it is intended that the “melted” or “liquid” lipids will be sufficiently liquid so that solids can be uniformly distributed through the lipid. It is assumed that those of ordinary skill in the art will understand that “solid” will mean solid-like and “melted” or “liquid” or “liquid-like” will mean sufficiently softened to be pourable.

Accordingly, it is an object of the invention to provide a single dosage multidrug composition having increased stability.

Another object of the invention is to provide a multidrug formulation that is effective against a variety of parasites.

Yet another object of the invention is to provide a single dosage multidrug formulation that is palatable to animals.

Another object of the invention is to provide a method for producing a multidrug composition with increased stability.

Other objects and features will be in part apparent and in part pointed out.

### DETAILED DESCRIPTION OF THE PREFERRED EMBODIMENTS

The invention provides an encapsulating system for isolating selected ingredients to prevent adverse interactions among those ingredients in a pharmaceutical formulation. In accordance with the invention, a multidrug composition comprising at least two active ingredients, and preferably, three or more active ingredients can be provided. Preferably, the composition comprises a combination of avermectins, hexahydropyrazinoisoquinolines and anthelmintic pyrimidines such as tetrahydropyrimidines. Preferred tetrahydropyrimidines include, for example, pyrantel pamoate, morantel and oxantel. Preferred hexahydropyrazinoisoquinolines include, for example, praziquantel. Other acceptable actives include benzazepines and salicylamides. Preferred avermectins include, for example, ivermectin, doramectin, selamectin and abamectin.

In one preferred embodiment of the invention, the multidrug composition comprises at least two active ingredients. In a preferred embodiment of the invention, the first active ingredient in the composition comprises a macrocyclic lactone, which is preferably ivermectin, and the second active ingredient comprises a hexahydropyrazinoisoquinoline, which is preferably praziquantel.

In another preferred embodiment of the invention, the multidrug composition comprises at least three active ingredients. The first active ingredient in the composition comprises a macrocyclic lactone such as ivermectin, selamectin, dormectin, moxidectin, eprinomectin, and abamectin, the second active ingredient comprises a tetrahydropyrimidine such as pyrantel pamoate, morantel and oxantel, and the third active ingredient in the composition comprises a hexahydropyrazinoisoquinoline, preferably, praziquantel.

In yet another preferred embodiment of the invention, the first active ingredient comprises ivermectin, the second active ingredient comprises pyrantel pamoate, and the third active ingredient comprises praziquantel.

The combination of avermectins, hexahydropyrazinoisoquinolines and anthelmintic pyrimidines target a wide variety of pathogenic organisms that can adversely affect the health of a mammal. However, administering three physically separate pharmaceutical compositions to an animal is undesirable and it has been determined that it would be beneficial to combine the ingredients into one formulation and in particular, into one tablet, capsule or packet containing a pharmaceutically effective amount of the active ingredients, thereby decreasing the number of administrations of formulations to the animal. Thus, when the active ingredients are combined into a single formulation, the formulation provides protection against a broader spectrum of parasites than a formulation containing any single parasitical agent. As used herein, the identification of an active ingredient, e.g., pyrantel pamoate or ivermectin, is intended to cover pharmaceutically active forms thereof such as salts, hydrochlorides, chelates, and so forth.

Several insecticides such as ivermectin, can adversely interact with other active ingredients, such as by producing an unstable composition. Therefore, the composition should be prepared in a manner so as to prevent ivermectin or other potentially troublesome ingredients from interacting with other active ingredients in the composition. While the present invention is directed to multidrug anthelmintic formulation comprising parasitical agents, this method may be used to prepare a composition containing other lipid compatible active ingredients that would typically interact with each other.

Accordingly, one embodiment of the invention is directed to a method of producing a stable multidrug composition that is effective against a variety of helminths. Preferred embodiments of the invention involve isolating the adversely reactive ingredients in a lipid base or separating the materials with layers of lipid instead. The use of lipid materials for forming solid-like pharmaceutical compositions is discussed in U.S. Pat. Nos. 6,541,025 and 6,340,471, the contents of which are incorporated herein by reference. Neither patent describe the procedure of using a lipid materials to separate active ingredients which will degrade one of the others. Rather, U.S. Pat. No. 6,340,471 is concerned with taste masking and controlled release during delivery, not preventing interaction of active ingredients.

In one preferred embodiment of the invention, a method for preparing a multidrug composition comprises the following steps:
(a) preparing a lipid suspension by mixing dried active ingredients into a mixture of melted lipids, wherein the dried active ingredients comprise a macrocyclic lactone, a tetrahydropyrimidine and a hexahydropyrazinoisoquinoline, and wherein at least one of the macrocyclic lactone and the hexahydropyrazinoisoquinoline is encapsulated or coated;
(b) pouring the liquid lipid suspension into a container at a temperature high enough to be pourable; and
(c) permitting the lipid suspension to cool, thereby forming a solid composition.

The first step in this method of preparing the multidrug composition includes encapsulating at least one of the active ingredients. A preferred embodiment of the invention is directed to an improved anthelmintic composition comprising ivermectin, pyrantel pamoate and praziquantel; however, any active ingredient that can exist as a dry particle can be used in this method for preparing a multidrug composition.

Several methods of encapsulation are commonly known to a skilled artisan and can be used in the present invention. In a preferred embodiment, the active ingredient, which preferably comprises ivermectin or praziquantel or both, is coated by a polymer such as ethyl cellulose using a pan coating technique.

In another embodiment of the invention, encapsulation of the active ingredients is achieved by spray granulation, which involves the drying of liquid (i.e., solution, suspension melt an so forth) while simultaneously building particle size. Methods of spray granulation are disclosed in PCT application no. 025,005, the contents of which are incorporated herein by reference. By mixing the active ingredient with a carrier in the liquid phase, the active ingredient can become “encapsulated” or substantially covered in a matrix of carrier after the granulation process. Granulation can be generally performed by spraying liquid into the fluidized powder and the granules are subsequently dried with heated air.

The active ingredients of the composition which are not intended to be encapsulated can be dry-blended to form dry particles of active ingredient. In a preferred embodiment of the invention, praziquantel is encapsulated or coated so to prevent contact with ivermectin and to mask its bitter taste when consumed by an animal.

In a preferred embodiment of the invention, lipids are used as the carrier vehicle for the active ingredients. The lipids are liquidified by heating to the appropriate temperature, preferably below the decomposition temperature of the active ingredients that are to be mixed into the liquidified lipid. The source of lipids can consist of a single component “hard butter”, which refers to a lipid system that has characteristics and/or a solid fat melting index similar to cocoa butter and is similar in rapid meltdown characteristics. Typical lipids include, but are not limited to, partially hydrogenated vegetable oil, soybean oil, cottonseed oil, palm oil and palm oil and palm kernel oil. The lipid system could also consist of petroleum wax, vegetable or animal stearines, or a high solids sharp melting point vegetable fat, or also combinations of hard butters and stearines. It is also possible to use mineral oil or petrolatum as the liquid hydrophobic system. The lipid base should have a melting point of about 80 to 130° F. In one embodiment of the invention, a surfactant is used to enhance the wetting of the dry particles. When used, the surfactant should be included of about 0.1 to 1.0% of the formulation and preferably, about 0.5%. However, surfactants need not be used.

In an alternative embodiment of the invention, matrix materials other than lipids can be used as the carrier vehicle for the active ingredient. For example, the carrier system can be prepared using gel-like polymers such as gelatin, agar, carrageenan and gellan, all of which can be dissolved in water by heating, and solidified to a gel by cooling. Alternatively, alginate solutions can also be used, which can be solidified to a gel by the addition of calcium (Ca²⁺) ions. This gelation can be controlled in terms of time to gel by the incorporation of the appropriate amounts of phosphate and calcium ions. Alginate solutions can be utilized as the carrier vehicle without the use of heat, therefore, alginate solutions are useful for active ingredients that are heat labile or thermally sensitive.

The dried ingredient mixture, which includes the dry and encapsulated active ingredients, and any fillers, optional flavorings and additives, are added to the heated lipid base to produce a pourable lipid suspension. The flavoring additives may include, for example, cheese, peanut better, ground animal protein, synthetic flavoring, and/or flavor enhancers such as monosodium glutamate. The range of flavoring agents or flavor enhancers may range from 10% to 19%.

In a preferred embodiment of the invention, the dried ingredient mixture is added to the melted lipid solution by simple mixing in order to uniformly distribute the active ingredient throughout the liquid lipid suspension. The dried ingredient mixture is added slowly in order to produce an advantageously homogeneous liquid lipid suspension, i.e., no agglomerated clumps of active ingredient should be formed in the lipid suspension. Preferably, this mixing step is accomplished in a heated mixing device that insures thorough mixing of all materials.

Optionally, once the dry particles of active ingredient are fully dispersed in the liquid lipid mixture, the first liquid lipid suspension is cooled to a temperature just slightly higher than the solidification point of the lipid base. The suspension should remain sufficiently liquid so that the suspension can be poured.

The pourable lipid suspension containing the suspended dry and encapsulated active ingredients is then poured into a mold or container for production into a final dosage form. After it is poured into the container, the liquid lipid layer containing the dried and encapsulated active ingredients is allowed to cool, thereby forming a solid composition.

In another preferred embodiment of the invention, the multidrug composition comprises ivermectin, pyrantel pamoate and praziquantel, wherein (i) at least one of the active ingredients, preferably ivermectin or praziquantel or both ivermectin and praziquantel, is encapsulated, and (ii) the ivermectin and praziquantel are contained in separate layers of the final solid composition. In another preferred embodiment of the invention, pyrantel pamoate is contained in a layer separate from, and in between each of the layers containing ivermectin and praziquantel, which provides an additional physical barrier to prevent ivermectin from interacting with praziquantel.

One preferred method of preparation of the multidrug composition comprises:
(a) preparing a first and second and optionally, a third lipid suspension comprising a first, second, and third active ingredient, respectively, at a temperature high enough to maintain the suspension in a pourable condition, wherein at least one of the active ingredients is encapsulated;
(b) pouring the first lipid suspension into a container;
(c) permitting the first lipid suspension to cool, thereby resulting in a first solid layer comprising the first active ingredient;
(d) dispersing the second lipid suspension at a temperature high enough to be pourable over the first solid layer whereby at least a portion of the surface of the first solid layer is covered by the second lipid suspension;
(e) permitting the second lipid suspension to cool, thereby resulting in a second solid layer located over the first solid layer;
(f) optionally pouring the third lipid suspension at a temperature high enough to be pourable over at least a portion of the top of the second solid layer; and
(g) allowing the third lipid suspension to cool, thereby resulting in a third solid layer located on top of the second solid layer, wherein the first active ingredient in the first solid layer is separated from the third active ingredient in the third solid layer by the second solid layer.

The first step in the process of preparing the multidrug composition includes preparing a first lipid suspension comprising a first active ingredient, preferably ivermectin. It should be understood that a second and third liquid lipid suspension comprising a second and third active ingredient, preferably pyrantel pamoate and praziquantel, respectively, is prepared using the same method. In one preferred embodiment of the invention, the dried active ingredient, ivermectin, pyrantel pamoate or praziquantel, is dry-blended with flavorings that makes the final dosage form taste and smell appealing to animals.

In a preferred embodiment of the invention, at least one of the active ingredients is encapsulated in the multidrug composition and preferably, the encapsulated active ingredient comprises ivermectin. Ivermectin is encapsulated using methods commonly known to one skilled in the art, or preferably, by pan coating or spray granulation, which are discussed herein.

The dried ingredient mixture, which includes the dry and encapsulated active ingredients, and any fillers, optional flavorings and additives, are slowly added with efficient mixing to the heated lipid base to produce an advantageously homogeneous liquid lipid suspension.

The same procedure is used to produce a second and third liquid lipid suspension comprising a second and third active ingredient, preferably pyrantel pamoate and praziquantel, respectively. In a preferred embodiment of the invention, praziquantel is encapsulated prior to mixing into the heated lipid base.

The first lipid suspension containing ivermectin is then poured into a mold or container for production into a final dosage form. After it is poured into the container, the first liquid lipid layer containing ivermectin is cooled to a solid. The second liquid lipid layer containing pyrantel pamoate is then poured on top of the first lipid layer containing ivermectin and cooled to a solid form. The temperature at which the first lipid suspension is poured over the first lipid layer should not be at such a high temperature that it melts the first lipid layer. Lastly, the third lipid layer containing praziquantel is poured on top of the second lipid layer containing pyrantel pamoate and cooled to a solid form.

It should be noted that the manner in which the second lipid layer is poured over the first lipid layer is important in the preparation of the composition of this embodiment of the present invention. The second lipid layer should be poured on top of the cooled first lipid layer in a manner so that once cooled, the second lipid layer advantageously covers the entire surface of the cooled first lipid layer in the container. After the second lipid layer is cooled to a solid, the third liquid lipid layer is poured on top of the second lipid layer and cooled to a solid. The second lipid layer contains pyrantel pamoate, which does not react with either ivermectin or praziquantel. Accordingly, in one embodiment of the invention, it is this second lipid layer that physically separates the first and third lipid layers and thereby prevents the interaction of ivermectin in the first lipid layer with praziquantel in the third lipid layer. Thus, an active-free second lipid layer can be used to separate a first active and a second active ingredient in a first and third layer, respectively.

In one preferred embodiment of the invention, the composition comprises ivermectin, pyrantel pamoate and praziquantel for the treatment of helminths. Preferably, the composition comprises approximately about 65 to 275 micrograms of ivermectin, 160 to 675 milligrams of pyrantel pamoate and 50 to 250 milligrams of praziquantel.

In one preferred embodiment of the invention, a composition comprising approximately 272 micrograms ivermectin, 656 milligrams pyrantel pamoate and 228 milligrams praziquantel can be administered to a dog weighing over 60 pounds.

In another preferred embodiment of the invention, a composition comprising approximately 136 micrograms ivermectin, 344 milligrams pyrantel pamoate and 240 milligrams coated praziquantel can be administered to a dog weighing between 20 and 60 pounds.

In another preferred embodiment of the invention, a composition comprising approximately 68 micrograms ivermectin, 164 milligrams pyrantel pamoate and 57 milligrams praziquantel can be administered to a dog weighing less than 20 pounds.

A preferred dosage of avermectin, e.g., ivermectin, is about 4-20 μg/Kg body weight of the animal administered monthly. It should be of course understood that the actual amount of ivermectin will vary depending upon the size of the animal being treated. In a preferred embodiment of the invention, the preferred dosage of ivermectin for dogs weighing 60 to 150 lbs (27-68 kg) is approximately 272 μg, the preferred dosage of ivermectin for dogs weighing 20 to 60 lbs (9-27 kg) is approximately 136 μg, and the preferred dosage of ivermectin for dogs weighing less than 20 pounds (less than 9 kg) is approximately 68 μg.

A preferred dosage of anthelmintic pyrimidines, e.g., pyrantel pamoate, is about 5-50 mg/Kg body weight administered monthly. A preferred dosage of hexahydropyrazinoisoquinaline, e.g., praziquantel, is about 3-19 mg/Kg body weight administered monthly. It should be of course understood that the actual amount of active ingredients will vary depending upon the size of the animal being treated.

The antiparasitic agents of this invention find their primary use in the treatment and/or prevention of helminthiasis; however, they may also be useful in the prevention and treatment of diseases caused by other parasites. Repeat treatments are given as required to combat re-infestations and are dependent upon the particular helminth. The techniques for administering these materials to compositions are known to those skilled in the field of veterinary medicine.

The preparations are suitable for combating pathogenic endoparasites which occur in animal husbandry and animal breeding in productive, breeding, zoo, laboratory, experimental animals and pets, and have a favorable toxicity to warm-blooded animals. In this connection, they are active against all or individual stages of development of the pests and against resistant and normally sensitive species. By combating pathogenic endoparasites, it is intended that disease, cases of death and reduction in production (for example in the production of meat, milk, wool, hides, eggs, etc.) are reduced so that more economic and simpler animal husbandry is possible by means of the use of the pharmaceutical formulation.

Productive and breeding animals include mammals, such as, for example, cattle, horses, sheep, pigs, goats, camels, water buffalo, donkeys, rabbits, fallow deer and reindeer, pelt animals, such as, for example, mink, chinchilla and raccoons, birds, such as, for example, chickens, geese, turkeys and ducks, fresh and salt-water fish, such as, for example, trout, carp and eels, and reptiles.

Laboratory and experimental animals include mice, rats, guinea pigs, golden hamsters, dogs and cats.

Pets include dogs and cats.

The formulation according to the invention is particularly preferably administered to dogs and cats, but is suitable for other mammals.

Administration can take place both prophylactically and therapeutically.

It is intended that the formulations be administered orally to the animal being treated.

Auxiliaries can include preservatives, antioxidants and colorants. Additional suitable auxiliaries can include lubricants, such as, for example, magnesium stearate, stearic acid, talcum and bentonites, disintegration-promoting substances, such as starch or transversely crosslinked polyvinyl pyrrolidone, binders, such as, for example, starch, gelatin or linear polyvinyl pyrrolidone, and dry binders, such as microcrystalline cellulose.

The formulation can also be in the form of a chewable, such as a beef-chewable containing ground or minced beef or other meat, in addition to other excipients listed above.

The materials in the final formulation, such as the excipients, auxiliaries, synergists and other materials, which aid in delivery, shelf-life, desired physical structure and so forth will be referred to herein generally as carrier material. As stated herein, carrier material could be pharmaceutically active under certain circumstances.

The following examples are given for purposes of illustration only and are not intended to be construed in a limiting manner.

### EXAMPLE 1

### Encapsulation or Coating of Ivermectin

5 grams of ethyl cellulose was dissolved in 100 ml of methylene chloride. 95 mg of ivermectin was placed in the rotating chamber of a pan coater. The ethyl cellulose in methylene chloride solution is sprayed slowly onto the ivermectin particles. Warm air was passed through the chamber to remove the solvent. The coated particles are stored in a suitable closed container.

### EXAMPLE 2

### Forming the Lipid Suspension

The lipids were placed into a kettle and heated in the range of about 90 to 120° F. and melted. The surfactant, lecithin, is added to the melted lipids with mixing, and the mixture is maintained at about 115° F.

The encapsulated ivermectin and pyrantel pamoate and praziquantel are dry-blended and slowly added incrementally to the melted lipid/surfactant mixture with mixing over a period of about 1 hour, to provide a smooth suspension with no lumps or agglomerations.

### EXAMPLE 3

### Molding the Multidrug Composition

A liquid lipid suspension containing ivermectin, pyrantel pamoate and praziquantel, was poured into a mold and cooled to a solid.

### EXAMPLE 4

### An Exemplary Formulation With Different Flavoring Agents

Table 1 shows an example of active ingredients in combination with different flavoring agents. The palatability of unflavored formulation is 55%. The addition of cheese, a combination of peanut butter and DDPE, or peanut butter only achieves 75%, 80%, and 65% palatability respectively.

TABLE 1

| | | | Peanut Butter + | | Peanut Butter | | | |
|---|---|---|---|---|---|---|---|---|
| | Cheese | | DDPE | | Only | | No Flavors | |
| | Weight | | Weight | | Weight | | Weight | |
| Ingredients | (mg) | % | (mg) | % | (mg) | % | (mg) | % |
| Ivermectin | 0.136 | 0.0068% | 0.136 | 0.0063% | 0.136 | 0.0069% | 0.136 | 0.0078% |
| Pyrantel pamoate | 344.00 | 17.200% | 344.00 | 15.897% | 344.00 | 17.515% | 344.00 | 19.770% |
| Coated praziquantel | 240.00 | 12.000% | 240.00 | 11.091% | 240.00 | 12.220% | 240.00 | 13.793% |
| Klx | 368.00 | 18.400% | 368.00 | 17.006% | 368.00 | 18.737% | 368.00 | 21.150% |
| Sterene 27 | 368.00 | 18.400% | 368.00 | 17.006% | 368.00 | 18.737% | 368.00 | 21.150% |
| Lecithin | 20.00 | 1.000% | 20.00 | 0.924% | 20.00 | 1.018% | 20.00 | 1.149% |
| Acdisol | 199.93 | 9.996% | 199.93 | 9.239% | 199.93 | 10.180% | 199.93 | 11.490% |
| Explotab | 199.93 | 9.996% | 199.93 | 9.239% | 199.93 | 10.180% | 199.93 | 11.490% |
| Cheese flavor | 40.00 | 2.000% | 0.00 | 0.000% | 0.00 | 0.000% | 0.00 | 0.000% |
| DDPE | 200.00 | 10.000% | 200.00 | 9.242% | 0.00 | 0.000% | 0.00 | 0.000% |
| Peanut butter flavor | 0.00 | 0.000% | 210.00 | 9.704% | 210.00 | 10.693% | 0.00 | 0.000% |
| Salt | 20.00 | 1.000% | 10.00 | 0.462% | 10.00 | 0.509% | 0.00 | 0.000% |
| Sucralose | 0.00 | 0.000% | 4.00 | 0.185% | 4.00 | 0.204% | 0.00 | 0.000% |
| Total | 1999.99 | 100.00% | 2163.99 | 100.00% | 1963.99 | 100.00% | 1739.99 | 100.00% |

| | | | | |
|---|---|---|---|---|
| Palatability % | 75 | 80 | 65 | 55 |
| (% of dogs consuming all the tablet) | | | | |

As shown in Table 1, the formulation may contain various flavoring agents. The DDPE is a flavor enhancer with registered trade name “Optimizor” from Applied Food Biotechnology, Inc., O'Fallon, Mo. This is a blend of vegetable derived ingredients that enhance palatability of formulations for dogs. The attached formulations contain cheese or peanut butter as flavorings. Other flavorings such as beef, poultry, pork, lamb, both synthetic and naturally-derived, or ground beef, chicken, turkey, pork and lamb may be included in the formulation. Flavor enhancers such as monosodium glutamate or other ground crunchy material such as kibble or ground biscuit may also be added. The range of flavoring agents or flavor enhancers may range from 10% to 19%.

It will thus be seen that the objects set forth above, among those made apparent from the preceding description, are efficiently attained and, since certain changes may be made in carrying out the above method and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall therebetween. Particularly it is to be understood that in said embodiments, ingredients or compounds recited in the singular are intended to include compatible mixtures of such ingredients wherever the sense permits.

What is claimed is:

1. A method for forming an anthelmintic formulation, comprising the steps of:
(a) combining a mixture of dried active ingredients of which at least one will degrade in the presence of at least one of others, comprising a macrocyclic lactone, a tetrahydropyrimidine and a hexahydropyrazinoisoquinoline with a composition comprising melted lipids, thereby forming a pourable active composition, wherein at least one of the macrocyclic lactone and the hexahydropyrazinoisoquinoline is present in the mixture as an encapsulated or coated particle;
(b) transferring the pourable active composition into a container;
(c) allowing the pourable active composition to cool and form a solid-like composition.

2. The method of claim 1, wherein the macrocyclic lactone comprises ivermectin.

3. The method of claim 2, wherein the tetrahydropyrimidine comprises pyrantel pamoate.

4. The method of claim 3, wherein the hexahydropyrazineisoquinoline comprises praziquantel.

5. The method of claim 4, wherein the encapsulated particles comprise ivermectin.

6. The method of claim 5, wherein the encapsulated particles comprise praziquantel.

7. The method of claim 1, wherein the encapsulated particles are prepared by spray granulation.

8. A pharmaceutical formulation comprising a lipid containing composition comprising at least two active ingredients that adversely react with each other, wherein at least one of the active ingredients is present in the formulation as encapsulated particles.

9. The pharmaceutical formulation of claim 8, wherein the at least one of the active ingredients comprise ivermectin, pyrantel pamoate and praziquantel.

10. The pharmaceutical formulation of claim 9, wherein the encapsulated particles comprise ivermectin.

11. The pharmaceutical formulation of claim 9, wherein the encapsulated particles comprise praziquantel.

12. The pharmaceutical formulation of claim 10, wherein the formulation further comprises at least two separate lipid containing compositions, wherein the first lipid composition comprises ivermectin and the second lipid composition comprises praziquantel.

13. The formulation of claim 10, wherein the formulation comprises at least about 65 to 275 micrograms of ivermectin.

14. The formulation of claim 13, wherein the formulation comprises about 160 to 675 milligrams of pyrantel pamoate.

15. The formulation of claim 14, wherein the formulation comprises about 50 to 250 milligrams of praziquantel.

16. The formulation of claim 15 wherein the formulation comprises about 68 micrograms of ivermectin, 164 milligrams of pyrantel pamoate and 57 milligrams of praziquantel.

17. The formulation of claim 15, wherein the formulation comprises about 136 micrograms of ivermectin, 344 milligrams of pyrantel pamoate and 240 milligrams of coated praziquantel.

18. The formulation of claim 15, wherein the formulation comprises about 272 micrograms of ivermectin, 656 milligrams of pyrantel pamoate and 228 milligrams of praziquantel.

19. A method for treating or preventing helminthiasis in mammals, said method comprising administering an anthelmintically effective amount of the composition of claim 1 to the mammal.

20. The method of claim 19, wherein the mammal is a cat or a dog.

21. A method for treating or preventing helminthiasis in mammals, said method comprising administering an anthelmintically effective amount of the multidrug composition of embodiment 5 to the mammal.

22. The method of claim 21, wherein the mammal is a cat or a dog.

23. A method for treating or preventing helminthiasis in mammals, said method comprising administering an anthelmintically effective amount of the composition of claim 15 to the mammal.

24. The method of claim 23, wherein the mammal is a cat or a dog.

25. A method for treating or preventing helminthiasis in mammals, said method comprising administering an anthelmintically effective amount of the multidrug composition of claim 16 to the mammal.

26. The method of claim 25, wherein the mammal is a cat or a dog.

27. A method for treating or preventing helminthiasis in mammals, said method comprising administering an anthelmintically effective amount of the multidrug composition of claim 17 to the mammal.

28. The method of claim 27, wherein the mammal is a cat or a dog.

29. A method for treating or preventing helminthiasis in mammals, said method comprising administering an anthelmintically effective amount of the multidrug composition of claim 18 to the mammal.

30. The method of claim 29, wherein the mammal is a cat or a dog.
